(19) United States
(12) Patent Application Publication
(10) Pub. No.: US 2008/0161705 A1
(43) Pub. Date: Jul. 3, 2008

(54) Devices and methods for ablating near AV groove

(76) Inventors: Jonathan L. Podmore, San Carlos, CA (US); Matthias Vaska, Palo Alto, CA (US)

Correspondence Address: SJM/AFD-WILEY, 14901 DEVEAU PLACE, MINNETONKA, MN, 55345-2126, US

(21) Appl. No.: 11/647,280

(22) Filed: Dec. 29, 2006

(51) Int. Cl. A61B 5/0402; A61N 7/00

(52) U.S. Cl. 600/509; 600/374; 600/437; 606/27

(57) ABSTRACT

The invention encompasses a device, system and methods for ablating tissue, where a ablating device includes an elongated shaft having a flexible distal end, and at least one ablation element and at least one electrode detecting electrical signals of the heart, such that a user can assess whether the at least one ablation element is located on a tissue of the atrioventricular (AV) groove of a patient's heart.

### BACKGROUND OF THE INVENTION

a. Field of the Invention

The instant invention generally relates to devices and methods for treating electrophysiological diseases of the heart. In particular, the instant invention relates to devices and methods for epicardial ablation for the treatment of atrial fibrillation.

b. Background Art

It is well known that atrial fibrillation results from disorganized electrical activity in the heart muscle (the myocardium). Procedures for treating atrial fibrillation may involve the creation of a series of elongated transmural lesions—that is, lesions extending through a sufficient thickness of the myocardium to block electrical conduction—to create conductive corridors of viable tissue bounded by scar tissue. Such procedures may be performed from outside the heart (epicardial ablation) using devices introduced into the patient's chest. Various techniques may be used for the creation of epicardial transmural lesions, including, for example, cryogenic ablation, radio frequency (RF) ablation, laser ablation, ultrasonic ablation, and microwave ablation. Epicardial ablation devices and methods useful for creating transmural lesions for the treatment of atrial fibrillation have been described in U.S. Pat. No. 7,052,493 to Vaska et al., which is hereby expressly incorporated by reference as though fully set forth herein.

In performing epicardial ablations, it is generally considered most efficacious to include a transmural lesion isolating the pulmonary veins from the surrounding myocardium. The pulmonary veins connect the lungs to the left atrium of the heart, joining the left atrial wall on the posterior side of the heart. It is also considered desirable to perform linear ablation at the mitral isthmus, which is defined as extending from the lateral mitral annulus to the ostium of the left inferior pulmonary vein (LIPV). Studies have shown that catheter ablation of the mitral isthmus, in combination with pulmonary vein (PV) isolation, consistently results in demonstrable conduction block and is associated with a high cure rate for paroxysmal atrial fibrillation. In addition to isolating the pulmonary veins and ablating the mitral isthmus, it is considered desirable to ablate the neural pathways, or the ganglionated plexi, which are located on the epicardial surface of the right and left atria.

In performing epicardial ablations for the treatment of atrial fibrillation, it is not desirable to ablate the atrioventricular (AV) groove. The atrioventricular (AV) groove, also called the coronary sulcus, demarcates the border between the atria and the ventricles. Ablating the AV groove may cause additional arrythimias such as ventricular tachycardia.

### BRIEF SUMMARY OF THE INVENTION

It is therefore desirable to have improved methods and devices for epicardial ablation that provide a mechanism for locating the AV groove of a patient's heart in order to avoid ablating this region. It is further desirable to provide devices for ablation that are capable of locating the ganglionated plexi, such that transmural lesions may be formed at these locations.

The present invention meets these and other objectives by providing ablation devices and systems having one or more electrodes for determining the position of an ablating element with respect to an anatomical feature of the heart. In an aspect of the invention, methods are provided for the use of an ablation device or catheter containing one or more electrodes for sensing electrical events in the AV groove region of heart tissue. In one embodiment, multiple sensors are spaced, electrically isolated, and oriented on the device to allow the measurement of atrial and ventricular electrical events to map the location of the device and ablating surfaces while in contact with the heart. Thus, the sensors are spaced and placed on the device or catheter at desired point(s) near the distal end or ablating surfaces. In another embodiment, one or more pacing electrodes can be used for pacing a heart and permitting a user to assess the location of the ablation elements.

In yet another embodiment, a first electrode can be separated from a second electrode along a first axis of the device, and a third electrode element is spaced from the first and second electrodes along a second, orthogonal axis. The first, second, and third electrodes convey signals measured along the different axes between different pairs of the electrodes and the electrodes can continuously record multiple electrical events at different relative orientations at one location. The multiple electrodes on different axes essentially form an array of sensors to detect atrial and ventricular electrical impulses, for example, so that the user can determine which if any part of the array is contacting atrial tissue and which if any part of the array is contacting ventricular tissue. This information coupled with the positioning of the sensors on the array locates the ablating surfaces or distal end of the device with respect to the heart's electrophysiological features, similar to mapping techniques. For example, an array spanning the AV groove will detect atrial impulses at one end and ventricular impulses on another, and these impulses can be easily differentiated by techniques known in the art. Typically, these sensor electrodes are bipolar electrodes, but combinations with unipolar electrodes can also be used. Additionally or alternatively, one or more ring electrodes spaced away from sensor electrode(s) can be used. Also, as noted, pacing electrodes can be used. The recorded signals include the characteristic atrial and ventricular signals and other, smaller AV node bypass signals typically associated with the AV groove or other anatomical feature. In another example, the changes in measurements while moving the ablating surface of the ablation catheter on the epicardial surface of the heart allow the location to be ascertained with respect to the AV node, ganglionated plexi, or other anatomical or electrophysiological active areas of the heart. In this way, for example, the physician can avoid any ablation near or in the AV groove.

In preferred embodiments, the electrodes are positioned at the distal end of a device and in a desired location and orientation with respect to the ablating surfaces of the device. A hand grip is generally located at the most proximal end of the device.

In another aspect, the invention provides a system or method of measuring or sensing electrical impulses in heart tissue to avoid contacting the ablating surfaces with the AV groove during an ablating procedure. The preferred ablating surfaces include those having high intensity focused ultrasound cells to ablate tissue in a directional manner. The orientation and location of these ablating, ultrasound cells can be more advantageously manipulated according to the devices and methods of the invention. With the ablating surfaces and sensors in place against epicardial surfaces, multiple or continuous recording of electrical activity in the heart, and/or responses to pacing sensors, are analyzed by methods available in the art to indicate and/or display the position of the ablating surfaces relative to the AV groove or other heart tissue. For example, the location of the AV groove can be recorded based upon bypass electrical activity in the AV groove region, and/or proximity to the AV node can indicate position relative to the AV groove.

In another aspect, a device for ablating tissue includes an ablating element with an ablating surface, one or more sensors for measuring electrical impulses of the heart, and a recorder for recording and/or displaying the electrical impulses detected and conveying information on the position of the ablating surfaces relative to an anatomical feature of the heart. As noted above, the preferred anatomical features include the AV groove and ganglionated plexi.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 illustrates an ablation device according to one embodiment of the present invention.

FIG. 2 depicts an ablation catheter with an electrode located proximally of the ablation elements.

FIG. 3 illustrates an ablation catheter with electrodes located distally of the ablation elements.

FIG. 4 depicts a device having two electrodes.

FIG. 5 schematically illustrates an ablation system incorporating a device according to the present invention.

### DETAILED DESCRIPTION OF THE INVENTION

As used herein, the words “preferred,” “preferentially,” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention and no disclaimer of other embodiments should be inferred from the discussion of a preferred embodiment or a figure showing a preferred embodiment.

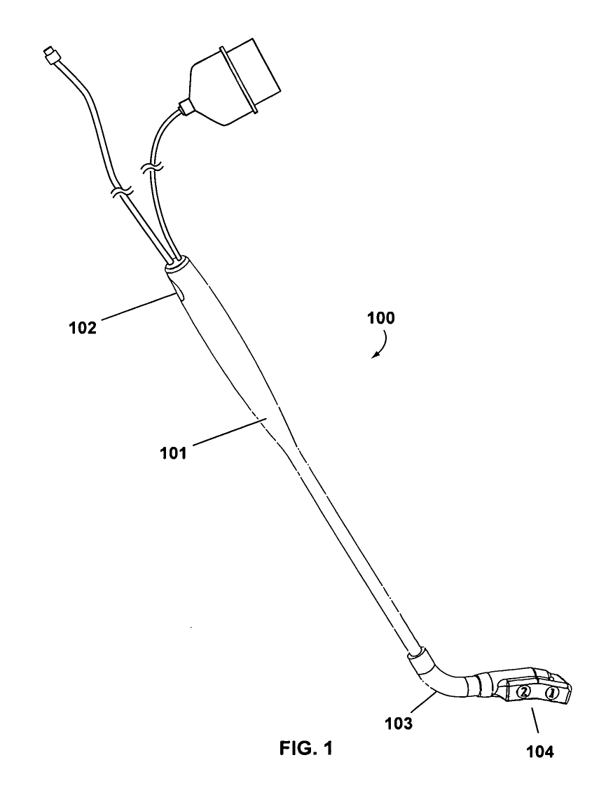

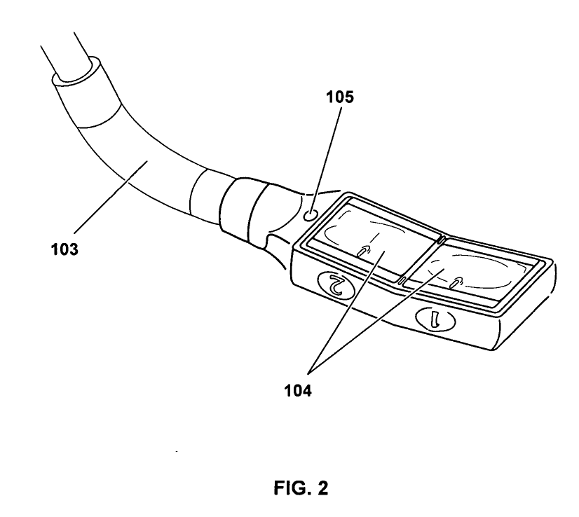

Referring to FIGS. 1 and 2, an ablation device according to one embodiment of the present invention is shown. Device 100 includes an elongated, rigid shaft 101 having a proximal end 102 and a flexible distal end 103. In particularly preferred embodiments, the device 100 has at least one ablation element, and preferably two ablation elements 106. The ablation device may, of course, have more or less than two ablation elements. The device also includes at least one electrode 105, such as a pacing electrode, located along the distal end of the shaft 101 at a desired position and orientation relative to the ablation elements 106 and particularly the ablating surfaces 104. The device may include more than one electrode or pacing electrode, be electrodes of different designs than shown in the figures, and in different positions than shown in the figures. As noted above, the electrodes can be positioned along one or more axes in the device.

In a preferred embodiment, the device 100 of FIG. 1 is a hand-held device that is capable of pacing and/or mapping cardiac tissue. The distal end of the shaft can be shaped by a user into a variety of positions to accommodate the angle of introduction of the ablating elements and ablating surfaces 104 of the elements, into the patient and the target surface orientation. The device preferably contacts a patient's heart tissue directly and is inserted through one or more openings in the patient's chest, such as a thoracotomy, a stemotomy, or a small incision.

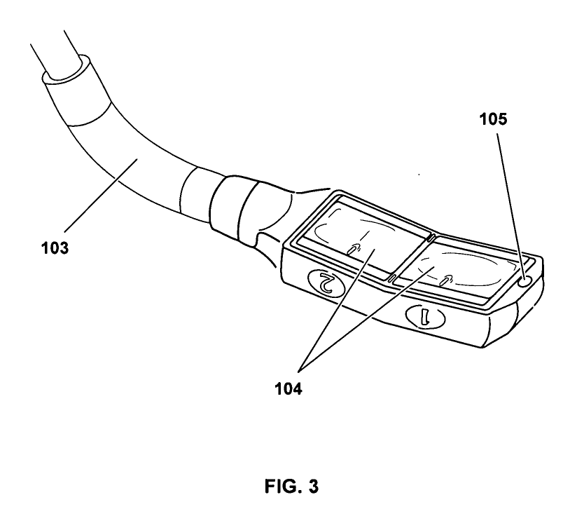

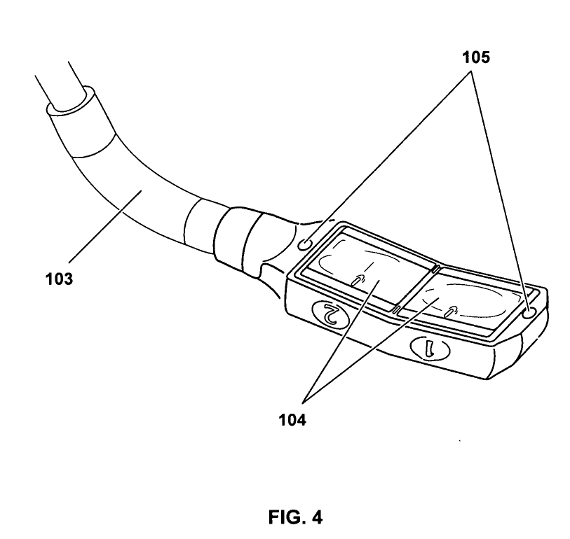

In preferred embodiments, an electrode 105 is located adjacent ablating surfaces 104, proximally as shown in FIG. 2, distally as shown in FIG. 3, or in multiple location as shown in FIG. 4. However, it should be understood that electrodes 105 may be located at any location along the distal end of the shaft in proximity to ablating surfaces 104, and in some embodiments two or more electrodes are located along a single or first axis and/or one or more additional electrodes along a separate, second axis, such as a second orthogonal axis to the first axis (not shown).

In the embodiments where a pacing electrode is used to deliver a pacing signal to a patient's heart, the position of the electrode should allow a pacing signal to cause the heart to react, which creates a distinctive signature in the electrophysiology data being detected or monitored relative to the location of the pacing electrode. By analyzing the electrophysiology data, a user can identify the approximate location of particular anatomical structures, such as the AV groove, based on signals sent to the tissue by a pacing electrode. For example, because the frequency of the pacing signal is typically twice that of a normally operating heart, the electrophysiology data will produce a distinctive spike or peak in a region indicative of the ventricles when the pacing signal is delivered to the AV groove. Similarly, bipolar or unipolar electrodes, or combinations of bipolar and unipolar electrodes, can detect electrical impulses from the heart and differences in the impulses can be recorded at different positions on the heart. The strength or amplitude of the signals detected can be correlated into a position relative to the AV node, for example, which then can indicate the position of the AV groove and ganglionated plexi.

Additionally or alternatively, electrocardiogram (ECG) data of a normal heartbeat and the respective P wave corresponding to the electric current caused by contraction of the atria compared to the electric current caused by contraction of the ventricles can be compared to indicate location on the heart. If a pacing electrode is used, a pacing signal delivered to a tissue defining the AV groove will result in an apparent response.

In some embodiments, electrodes are positioned sufficiently close to ablating surfaces such that the location of ablation surfaces can be identified with respect to the location of at least one of the electrodes.

Preferably, ablating surfaces 104 are from ultrasonic ablation elements, as described in the art or in U.S. Pat. No. 7,052,493, incorporated herein by reference. However, the ablation elements may be any suitable ablation elements, such as radio frequency (RF) elements, laser elements, cryogenic elements, or microwave elements. The ablating elements may be fixed relative to one another, or, alternatively, may have a flexible or malleable connection therebetween in order to adjust the relative orientation or position of ablating surfaces 104 relative to tissue. It is preferred to vary the frequency of the energy delivered to the ablating elements when ablating the tissue; however, the ablation elements may, of course, be operated at a single frequency. Various treatment methods for delivering energy to the ablation elements are described in U.S. Pat. No. 7,052,493. In a first treatment method, the ablation elements are activated at a frequency of about 2 MHz to about 7 MHz, and preferably of about 3.5 MHz, and a power of about 80 watts to about 150 watts, and preferably of about 130 watts, in short bursts. Following treatment at the first frequency, the ablation elements are preferably operated at a frequency of about 2 MHz to about 14 MHz, more preferably about 3 MHz to about 7 MHz, and most preferably about 6 MHz, and a power of about 20 watts to about 80 watts, and preferably about 60 watts. As a final treatment, the ablation elements are preferably operated at a frequency of at least about 3 MHz to about 16 MHz, and preferably at about 6 MHz. In a preferred method, the ablation elements are operated at about 2 watts to about 20 watts, and more preferably about 15 watts.

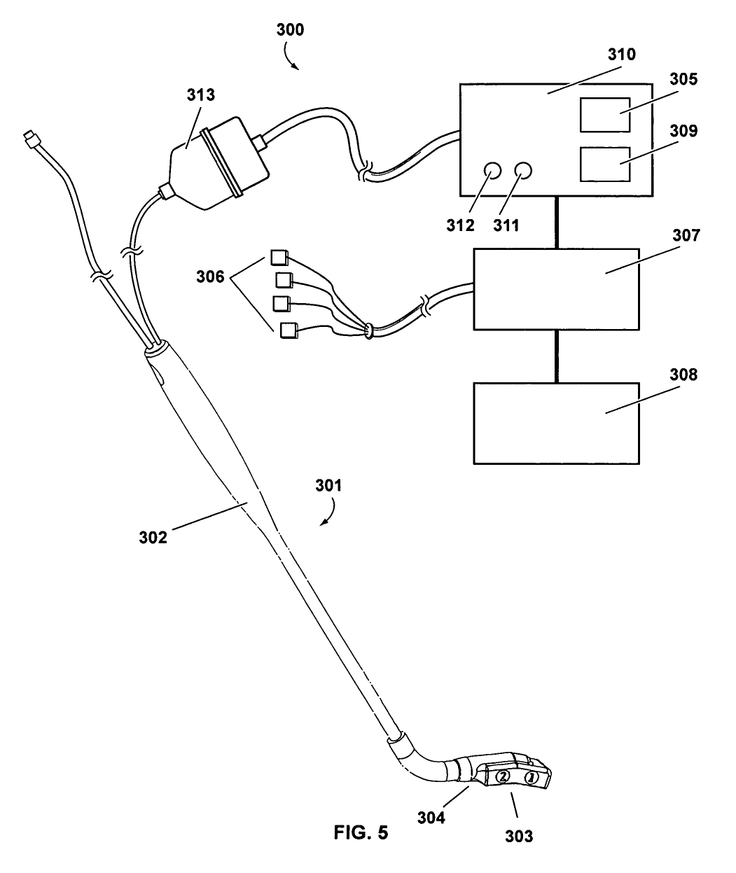

Referring to FIG. 5, a system for ablating cardiac tissue is shown. The system includes an ablation device 301, such as, for example, the ablation device described above with reference to FIGS. 1 to 4, or any other suitable ablation device. The ablation device 301 includes an elongated shaft 302, at least one ablation element 303 and at least one pacing electrode 304 for delivering a pacing signal to a cardiac tissue. System 300 also includes a generator 305, at least one measurement electrode 306, a data capture device 307 and a monitor 308. Generator 305 is coupled to the at least one pacing electrode 304, for example, via a plug 313. Generator 305 generates a pacing signal that is delivered to the tissue via pacing electrode 304. The pacing signal is preferably about 1.0 to 3.0 Hz, more preferably about 2.0 Hz, and preferably about 1 to 15 volts, more preferably about 10 volts.

In preferred embodiments, the system includes a plurality of measurement electrodes 306 for measuring electrophysiological data of a patient's heart. Measurement electrodes 306 measure electrophysiological data of a patient's heart, such as, for example, electric current or electric voltage data. The electrophysiological data is stored in a data capture device 307. The stored data may then be displayed on monitor 308. The electrophysiological data enables a user to assess a location of pacing electrode 304. By way of example only, the electrophysiological data may be electric voltage data displayed on an electrocardiograph ECG. If the ECG confirms a response in the ventricle after a pacing signal is delivered to the tissue, this indicates that the pacing electrode is located on the AV groove. If the ECG does not indicate a response in the ventricle, the pacing electrode is not located on the AV groove.

The system may also include a signal analyzer 309. Signal analyzer 309 analyzes the electrophysiological data measured by the measurement electrodes 306 to assess whether the AV groove has been stimulated. When pacing electrode 304 stimulates a tissue of the AV groove, the signal analyzer 309 generates a first indicator signal 311 to notify the user of the location of the pacing electrode. The first indicator signal 311 may be a light or an audible signal such as a beep. The signal analyzer 309 may generate a second indicator signal 312, distinct from the first indicator signal 311, to indicate when the pacing electrode 304 is not positioned on the AV groove. For example, the first indicator signal 311, indicating that the pacing electrode is positioned on the AV groove, may be a red light, and the second indicator signal 312, indicating that the pacing electrode is not positioned on the AV groove, may be a green light. Any type of signal or combination of signals can be used to indicate the location of the pacing electrode with respect to the AV groove, or other anatomical or electrophysiological feature of the heart.

The system 300 may further include a controller 310. The controller 310 automatically generates a signal to inhibit the ablation device from performing an ablation when the first indicator signal 311 is activated, indicating that the pacing electrode is positioned on the AV groove. To ensure the AV groove tissue is not ablated, the controller prevents the activation of the ablation device when the signal analyzer generates a signal indicative of the pacing electrode having delivered a pacing signal to the AV groove. When the pacing electrode is not positioned on the AV groove, as indicated by the second indicator signal 312, the controller automatically enables the ablation device so the tissue can be ablated. The disabling feature serves as a safeguard to ensure the ablation device cannot be activated to ablate tissue when the pacing electrode is positioned on the AV groove.

A method of ablating cardiac tissue is now described. An ablation device having at least one ablation element and at least one pacing electrode, such as, for example, the ablation device described herein with reference to FIGS. 1 and 2, or any other suitable ablation device, is provided. The ablation elements and pacing electrode are located at a distal end of the device. The distal end of the ablation device is placed on an epicardial surface of a patient's heart such that the ablation elements and pacing electrode are positioned at a first location on the epicardial surface. A plurality of measurement electrodes are placed on an external surface of the patient's body. The measurement electrodes measure electrophysiological data, such as, for example, electric voltage or electric current of the heart. A pacing signal is applied to the tissue via the pacing electrode, and the measurement electrodes detect and measure the electrophysiological data of the patient. The electrophysiological data may be monitored manually, for example, by a physician observing a patient's ECG, or the data may be monitored electronically, for example, using a signal analyzer.

The physician may analyze the electrophysiological data to determine whether the pacing electrode is positioned on the AV groove. If the data indicates that the pacing electrode is positioned on the AV groove, the distal end of the ablation device is moved to a second location on the epicardial surface, a pacing signal is again applied, and the electrophysiological data is observed and analyzed. When the data indicates that the pacing electrode is not positioned on the AV groove, the tissue is then ablated.

Alternatively, a signal analyzer may generate an indicator signal to indicate when the pacing electrode is positioned on the AV groove. The physician may observe the indicator signal to determine whether the pacing electrode is positioned on the AV groove. When the signal analyzer generates a signal indicative of the pacing electrode being positioned on the AV groove, the ablation device is moved to a second location on the epicardial surface of the patient's heart and a second pacing signal is delivered to the tissue. When the signal analyzer generates a signal indicative of the pacing electrode not being positioned on the AV groove, the tissue is ablated.

In another embodiment where a high intensity ultrasound ablation element or elements, as known in the art, are used at the distal end of the device, an array of sensor electrodes can be disposed on the distal end, with each electrode at a specific location relative to the ablating surface(s). The use of an array of sensor electrodes allows the physician to determine when, for example, the ablating surfaces are spanning the AV groove through the detection of both atrial and ventricular electrical impulses. The array can be used in conjunction with moveable ablating elements, actuated by the physician to move along a set path once the ablating surfaces are positioned in a desired manner. Thus, for example, the system of the invention can map the location of the ablating surfaces and confirm that the AV groove will not be ablated during the movement of the moveable ablating elements. Available electrode arrays for mapping the electrophysiological features of the heart can be adapted and used in this manner with ablating elements also.

Although several embodiments of this invention have been described above with a certain degree of particularity, those skilled in the art could make numerous alterations to the disclosed embodiments without departing from the spirit or scope of this invention. For example, although ablation device 100 has been described with respect to pacing a heart to identify the location of the AV groove, ablation device 100 may be used to identify additional anatomical structures, such as the ganglionated plexi, including the anterior and superior right ganglionated plexi, the anterior and superior left ganglionated plexi, the SVC-RA ganglionated plexus and the crux ganglionated plexus.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A device for ablating epicardial tissue having a proximal end and a distal end, the device comprising:
at least one ablation element having an ablating surface, the ablation element disposed along the distal end; and
at least one pacing electrode disposed along the distal end and at a location relative to the ablating surface of the ablation element, the pacing electrode capable of delivering a pacing signal to a cardiac tissue wherein the pacing signal can be detected and analyzed to indicate the position of the ablating surface relative to an anatomical feature on the epicardial tissue.

2. The device of claim 1, wherein the at least one ablation element is a high intensity focused ultrasound element.

3. The device of claim 1, having two ablation elements.

4. The device of claim 1, having multiple pacing electrodes positioned along the distal end.

5. The device of claim 1, wherein the at least one pacing electrode is positioned proximally of the at least one ablation element.

6. A device for ablating epicardial tissue, comprising:
a shaft having a proximal end and a distal end;
at least one ablation element having an ablating surface, the ablation element disposed along the distal end; and
two or more electrodes disposed along the distal end and at desired locations relative to the ablating surface of the ablation element, the electrodes capable of detecting electrical signals in cardiac tissue, wherein the signals can be analyzed to indicate the position of the ablating surface relative to an anatomical feature on the epicardial tissue.

7. The device of claim 6, wherein the at least one ablation element is a high intensity focused ultrasound element.

8. The device of claim 6, having two ablation elements.

9. The device of claim 6, wherein the two or more electrodes are a first and second electrode positioned to define a first axis, and further comprising at least a third electrode disposed along a second axis.

10. A system for ablating tissue comprising:
an ablation device having a shaft with proximal and distal ends, at least one ablation element and at least one pacing electrode for delivering a pacing signal to a cardiac tissue at the distal end;
a generator for generating a pacing signal, the generator being coupled to the at least one pacing electrode;
at least one measurement electrode for measuring electrophysiological data of a patient's heart; and
a monitor to present the electrophysiology data to a user,
wherein the monitor enables a user to assess a location of the at least one pacing electrode when a pacing signal is delivered to the patient's heart.

11. The system of claim 10, further comprising a signal analyzer, wherein the signal analyzer analyzes the electrophysiological data and generates an indicator signal indicative of the at least one pacing electrode having delivered a pacing signal to an atrioventricular groove of the patient's heart.

12. The system of claim 10, further comprising a data capture device for storing the patient's electrophysiology data measured by the at least one measurement electrode.

13. The system of claim 11, further comprising a controller, wherein the controller automatically disables the ablation device in response to an indication by the indicator signal that the at least one pacing electrode delivered a pacing signal to an atrioventricular groove of a patient's heart.

14. The system of claim 10, wherein the ablation device has two ablation elements and one pacing electrode.

15. The system of claim 10, wherein the ablation elements are high intensity focused ultrasound elements.

16. The system of claim 10, having a plurality of measurement electrodes.

17. A method of ablating cardiac tissue, comprising:
providing an ablation device having a shaft with proximal and distal ends, the distal end having at least one ultrasound ablation element and at least one electrode adjacent the at least one ultrasound ablation element;
placing the distal end of the ablation device at a first location on an epicardial surface of a patient's heart such that the at least one ablation element and the at least one electrode are positioned at the first location on the epicardial surface;
detecting a first electrical signal from the heart at the first location on the epicardial surface of the patient's heart via the electrode and mapping that location by measuring atrial or ventricular electrical impulses;
ablating tissue outside the atrioventricular (AV) groove of the heart by activating the at least one ultrasound ablation element.

18. The method of claim 17, wherein multiple electrodes are positioned adjacent at least one ultrasound ablation element.

19. The method of claim 17, further comprising
moving the distal end of the ablation device to a second location on an epicardial surface of the patient's heart;
detecting a second electrical signal from the heart at the second location on the epicardial surface of the patient's heart via the electrode; and
comparing the first and second signals to map the location of the electrode relative to the atrium, ventricle, or AV node of the patient's heart.

20. The method of claim 18, wherein the multiple electrodes are formed into an array to map the location of the ends of the array relative to atrial and ventricular tissue.

21. A method of locating an atrioventricular (AV) groove of a patient's heart, comprising:
providing an ablation device having a shaft with proximal and distal ends, the distal end having at least one ablation element and at least one pacing electrode adjacent the at least one ablation element;
placing the distal end of the ablation device on an epicardial surface of the patient's heart;
applying a pacing signal to the epicardial surface of the patient's heart;
measuring electrophysiological data of the patient; and
monitoring the electrophysiological data to determine whether the ablation device was placed on the atrioventricular (AV) groove of the heart.

22. The method of claim 21, wherein the monitoring step comprises observing the electrophysiological data on a monitor.

23. The method of claim 21, wherein the monitoring step comprises observing a signal generated by a signal analyzer.
